(19) United States
(12) Patent Application Publication
(10) Pub. No.: US 2020/0158584 A1
(43) Pub. Date: May 21, 2020

(54) PRESSURE SENSING TEXTILE

(71) Applicant: THE UNIVERSITY OF NOTTINGHAM, Nottingham Nottinghamshire (GB)

(72) Inventors: Stephen MORGAN, Nottingham Nottinghamshire (GB); Serhiy KORPOSH, Nottingham Nottinghamshire (GB); Barrie HAYES-GILL, Nottingham Nottinghamshire (GB); Ricardo Nuno Goncalves CORREIA, Nottingham Nottinghamshire (GB)

(21) Appl. No.: 16/686,878

(22) Filed: Nov. 18, 2019

Related U.S. Application Data
(63) Continuation of application No. PCT/GB2018/051321, filed on May 16, 2018.

(30) Foreign Application Priority Data
May 16, 2017 (GB) 1707831.2

(51) Int. Cl. G01L 1/24; D03D 1/00; G02B 6/12; A61B 5/00

(52) U.S. Cl. CPC G01L 1/246; D03D 1/0088; A61B 5/6802; G02B 2006/12138; G02B 6/12

(57) ABSTRACT

A textile comprising a pressure sensor, the pressure sensor comprising an optical fibre (102) with a transducer FBG (107), and a transducer patch (101) having a first face and a second opposite face, wherein: the transducer FBG (107) is embedded in the transducer patch (101), the transducer patch (101) is less than 10 mm in length along the optical fibre (102), and the transducer patch (101) is configured to cause longitudinal strain in the transducer FBG (107) in response to the transducer patch (101) being subject to a pressure load on the first and second face, the longitudinal strain arising as a result of extension of the transducer patch (101) due to the Poisson effect.

The invention relates to a pressure sensing textile, and to a method of making and using a pressure sensing textile.

Textiles that include a means of pressure sensing are useful in a number of applications. One application is in compression bandages. The aim of a compression bandage is improve venous return and to reduce oedema. It is well recognised that appropriate interface pressure between the compression bandage and the skin is crucial for optimal healing. Several devices have been developed to achieve this and are usually based on inflating a bladder placed within the layers of bandage and measuring the air pressure in the bladder using a manometer (Partsch, H., and G. Mosti. “Comparison of three portable instruments to measure compression pressure.” International angiology: a journal of the International Union of Angiology 29.5 (2010): 426-430.). However optical fibre sensing offers clear advantages as the textile based pressure sensors can be unobtrusively located within the bandage.

Fibre Bragg Gratings (FBGs) can be used to measure textile pressure in compression therapy (Wang, David Hsiao-Chuan, et al. “An optical fiber Bragg grating force sensor for monitoring sub-bandage pressure during compression therapy.” Optics express 21.17 (2013): 19799-19807). Wang et al disclose the use of a twisted pair of fibres, with FBGs in each fibre in the same position along the twisted pair. The twisted fibre pair is secured to a rigid disc structure over a cavity in the disc, with a first FBG uppermost, directly above the second (lower) FBG. This arrangement of FBGs results in a differential measurement, since the upper FBG will be compressed as a result of curvature of the twisted pair in response to downward pressure (toward the cavity), while the lower FBG will be extended. Common mode extension and compression, for example due to temperature and axial stress applied to the twisted pair, can be rejected from the measurement. Although this approach shows some promise, it is rather complex, and requires relatively precise alignment of each fibre pair in the rigid support disc.

A pressure sensing textile would also be useful in the context of other compression garments, such as those used in recovery from sports (e.g. shirts, shorts, leggings etc).

A simpler pressure sensing textile that offers at least one of reduced cost, greater robustness and improved integration is desired.

According to an aspect of the invention, there is provided a pressure sensor comprising a pressure sensor, the pressure sensor comprising an optical fibre with a transducer FBG (fibre Bragg grating), and a transducer patch having a first face and a second opposite face, wherein: the transducer FBG is embedded in the transducer patch, the transducer patch is less than 10 mm in length along the optical fibre, and the transducer patch is configured to cause longitudinal strain in the transducer FBG in response to the transducer patch being subject to a pressure load on the first and second face, the longitudinal strain arising as a result of extension of the transducer patch due to the Poisson effect.

According to a first aspect of the invention, there is provided a textile comprising a pressure sensor, the pressure sensor comprising an optical fibre with a transducer FBG (fibre Bragg grating), and a transducer patch having a first face and a second opposite face, wherein: the transducer FBG is embedded in the transducer patch, the transducer patch is less than 10 mm in length along the optical fibre, and the transducer patch is configured to cause longitudinal strain in the transducer FBG in response to the transducer patch being subject to a pressure load on the first and second face, the longitudinal strain arising as a result of extension of the transducer patch due to the Poisson effect. The small dimensions of the patch may mean that is it relatively insensitive to bending.

The patch may be 4 mm to 6 mm in length along the optical fibre.

The patch may have a width of less than 10 mm in length, or a width of 4 mm to 6 mm, or a width of from 0.5 mm to 6 mm or 2 mm to 6 mm.

The patch may have a thickness of between 0.5 mm and 3 mm.

The patch may comprise an epoxy resin material, or a polyepoxide material.

The optical fibre may comprise a strain compensation FBG that is not embedded in a patch, for providing compensation for a longitudinal force applied to the optical fibre.

The optical fibre may comprise a temperature compensation FBG and pressure insensitive patch, wherein:

the temperature compensation FBG is the last FBG before the end of the optical fibre,

the pressure insensitive patch comprises a substantially rigid support member with a cavity, and

the temperature compensation FBG is received in a clearance fit within the cavity so that the temperature compensation FBG is free to expand and contract independently of the rigid support member.

In some embodiments the temperature compensation FBG (within the cavity) may be for compensating for both temperature and axial strain. The temperature compensation FBG may not be the last FBG before the end of the fibre. The temperature compensation FBG may be placed inside a clearance fit cavity and the entrance and exit of the cavity glued (or otherwise attached) to the optical fibre.

The pressure insensitive patch may comprise the same material and dimensions as the transducer patch.

The pressure sensor may comprise a patch that includes a temperature compensation FBG in a rigid support member, as described above, and a transducer FBG.

The pressure sensor may comprise a plurality of transducer patches, and the optical fibre comprises a plurality of transducer FBGs, wherein each transducer FBG is embedded in a respective transducer patch, so as to provide a pressure sensor with a plurality of pressure transducers along an optical fibre.

The pressure sensor may further comprise a plurality of strain compensation FBGs, wherein each strain compensation FBG is not embedded in a patch and is configured to provide strain compensation for a longitudinal force applied to the optical fibre, each transducer FBG having a corresponding strain compensation FBG adjacent thereto.

Each strain compensation FBG may be within 2 cm, 1 cm, or 5 mm of the corresponding transducer FBG.

The optical fibre may be a high birefringent optical fibre, so that each transducer FBG has a spectral response that enables pressure to be distinguished from shear stress.

The textile may comprise a heat fusible yarn, and the optical fibre be attached to the fabric by a fused region of the heat fusible yarn.

The optical fibre may be integrated with the textile by knitting, weaving, embroidering or threading the optical fibre into the textile.

The optical fibre may be integrated with a textile by fusing between a first and second sheet of fusible yarn.

According to a second aspect of the invention, there is provided a compression bandage or compression garment comprising the textile according to the first aspect.

According to a third aspect of the invention, there is provided a sock, footwear or orthotic comprising the textile according to the first aspect.

According to a fourth aspect, there is provided a system for determining pressure on a textile in at least one location, comprising:

a textile according to the first aspect, wherein the optical fibre further comprises:
a strain compensation FBG that is not embedded in a patch, for providing compensation for longitudinal force applied to the optical fibre, and
an optical interrogator for determining a Bragg wavelength of the transducer FBG and strain compensation FBG;
a processor configured to determine a pressure load from the Bragg wavelength of the transducer FBG, including compensating for longitudinal force applied to the optical fibre.

The optical fibre may be curved in a zig-zag or oscillatory way, so that the optical fibre can accommodate stretch in the fabric by bending, without significant axial strain.

Compensating for a longitudinal force applied to the optical fibre may comprise subtracting the wavelength shift of the strain compensation FBG from the wavelength shift of the transducer FBG before determining the pressure load.

The textile may further comprise a temperature compensation FBG and pressure insensitive patch, wherein:

the temperature compensation FBG is the last FBG before the end of the optical fibre, the pressure insensitive patch comprises a substantially rigid support member with a cavity, the temperature compensation FBG is received in a clearance fit within the cavity so that the temperature compensation FBG is free to expand and contract independently of the rigid support member, and

the processor is configured to compensate for temperature using the Bragg wavelength of the temperature compensation FBG when determining the pressure load.

According to a fifth aspect of the invention, there is provided method of designing an item of footwear or an orthosis or a prosthesis, comprising:

a user performing activity while wearing the footwear or orthotic, and the footwear or orthotic is according to the third aspect, or wherein the user is wearing a sock according to the third aspect between the footwear or orthotic and the user's foot;

recording data from a plurality of transducer FBGs indicating at least pressure in a plurality of locations about the user's foot;

modifying a design of the item or footwear or the orthotic in response to the data.

Modifying the design may comprise identifying regions of high pressure, and modifying the design of the footwear or orthotic to reduce the pressure in the high pressure region.

The activity is a sporting activity, such as running (long distance, sprinting etc.), football, rugby, hockey, ice hockey, tennis, squash, badminton etc.

According to a sixth aspect, there is provided a method of checking the pressure applied by a compression bandage, comprising:

connecting an optical fibre of a compression bandage according to the second aspect to an optical interrogator;

using the optical interrogator to determine the interface pressure of the compression bandage in at least one location from the Bragg wavelength of at least one transducer FBG.

More generally, and according to a seventh aspect, there is provided a method of monitoring pressure applied to a portion of a user's body via a textile according to an aspect, comprising:

applying the textile to the user's body,

using an optical interrogator to determine a Bragg wavelength of at least one transducer FBG,

using a processor to determine at least one pressure load from the Bragg wavelength from the at least one transducer FBG.

The fifth and sixth aspects are specific examples of this more general aspect.

Applying the textile to the user's body may comprise at least one of a user: wearing the textile, lying on the textile, sitting on the textile, resting part of their body on the textile, and/or performing a work or leisure activity while at least part of their body is in contact with the textile.

The method may comprise using the optical interrogator to determine a Bragg wavelength of at least one strain compensation FBG and/or at least one temperature compensation FBG; and

using the processor to determine a pressure load from the Bragg wavelength of the transducer FBG, including compensating for longitudinal force applied to the optical fibre.

Compensating for a longitudinal force applied to the optical fibre may comprise subtracting the wavelength shift of the strain compensation FBG from the wavelength shift of the transducer FBG before determining the pressure load.

The textile may further comprise a temperature compensation FBG and pressure insensitive patch, wherein:

the temperature compensation FBG is the last FBG before the end of the optical fibre, the pressure insensitive patch comprises a substantially rigid support member with a cavity, the temperature compensation FBG is received in a clearance fit within the cavity so that the temperature compensation FBG is free to expand and contract independently of the rigid support member, and

the processor compensates for temperature using the Bragg wavelength of the temperature compensation FBG when determining the pressure load.

The features of each aspect may be combined with those of any other aspect or embodiment. Any of the methods may be performed using a textile or system according to another aspect. Any of the textile or system aspects or embodiments may be configured to perform any of the methods.

Example embodiments will now be described, with reference to the accompanying drawings, in which:

FIG. 1 is a schematic drawing of an FBG patch sensor according to an embodiment;

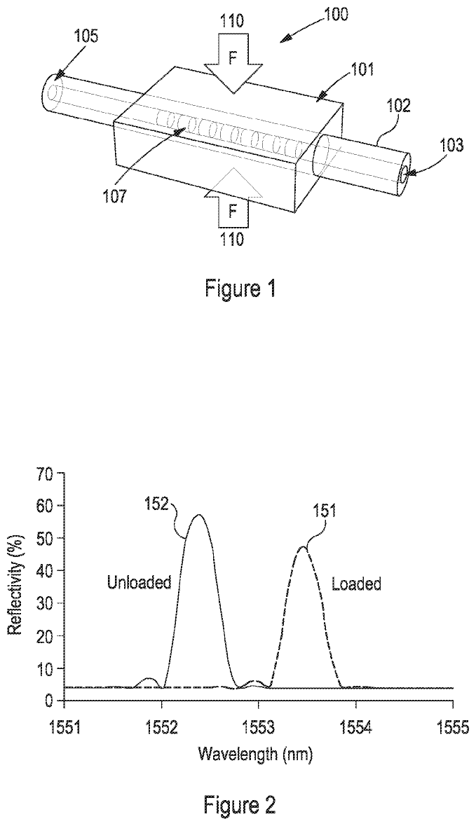

FIG. 2 is a graph illustrating the response of the patch sensor to a transverse load;

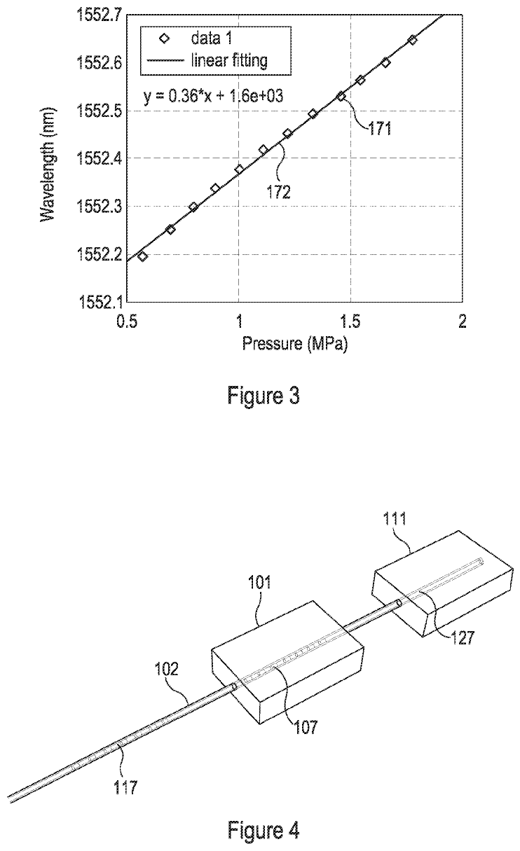

FIG. 3 is a further graph illustrating the linearity of the response of a patch sensor according to an embodiment;

FIG. 4 is a schematic of an optical fibre based pressure sensor according to an embodiment comprising an FBG pressure transducer patch, a strain reference grating and a temperature reference grating;

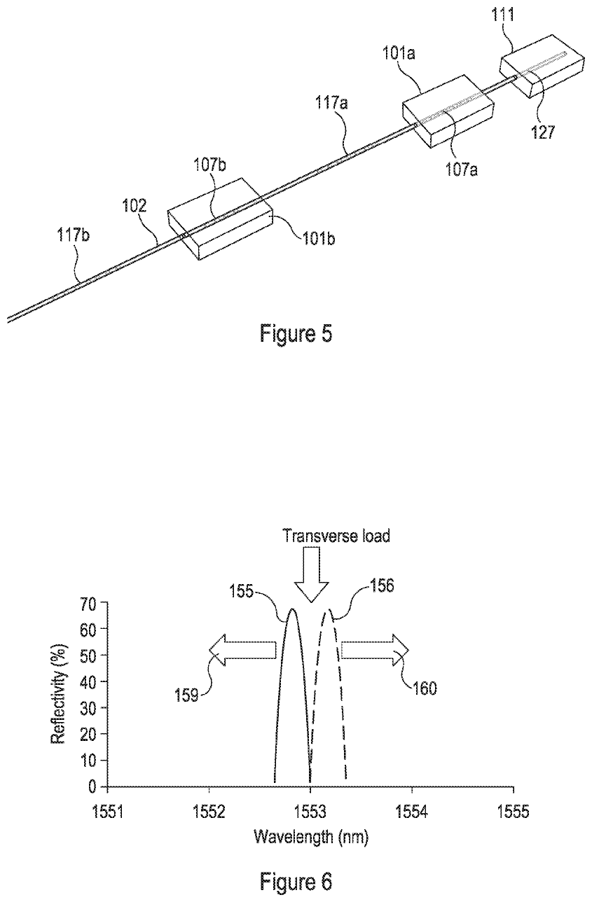

FIG. 5 is a schematic of an optical fibre based pressure sensor according to an embodiment comprising a first and second pressure transducer patch, a first and second strain reference grating, and a temperature reference grating;

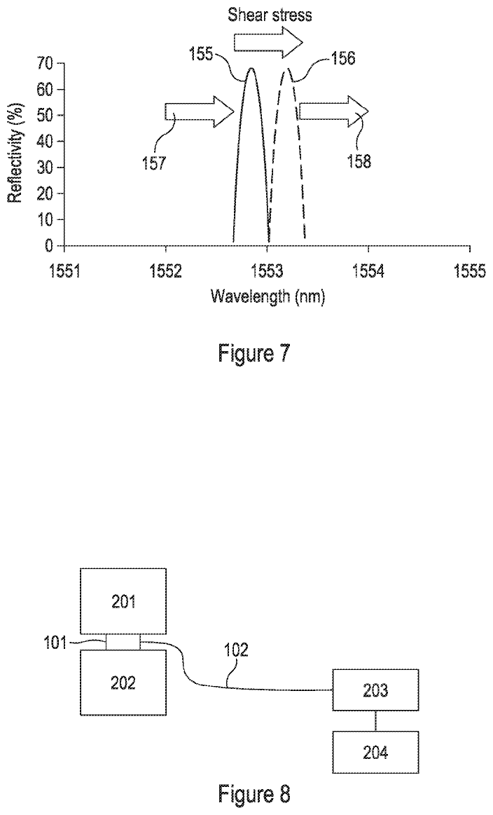

FIGS. 6 and 7 are graphs illustrating the response of an FBG sensor comprising a high birefringent fibre to a transverse load and shear load respectively, illustrating that such a sensor can distinguish between pressure and shear loading; and

FIG. 8 is a schematic of a test arrangement for characterising the response of a patch device to transverse loading.

Referring to FIG. 1 a pressure sensor is shown, comprising an optical fibre 102 embedded in a patch 101.

The fibre 102 comprises a fibre Bragg grating (FBG) 107, in which the refractive index of the fibre has a periodic variation (e.g. written by masking or interference of a UV laser). A FBG has a characteristic spectral response, defined by the periodic variation in refractive index or grating period. FBGs typically have a very narrow spectral peak in reflectance corresponding with the grating period. Longitudinal strain in the fibre will change the grating period and effective refractive index of the core mode, resulting in a change in the wavelength of the spectral peak, which can be detected with a high degree of precision. When the FBG is illuminated with a broadband optical source, a narrow-band spectral component will be reflected from the FBG, while other wavelengths outside this band will be transmitted. The wavelength of this reflectance peak may be termed the Bragg wavelength. The Bragg wavelength is linearly dependant on the grating period and effective refractive index. The change in Bragg wavelength can be used to infer the pressure load exerted on the patch. The FBG may be between 1 mm and 4 mm long.

The patch 101 may be formed of any suitable material, such as a polymer, or more specifically an epoxy resin. The patch may have a length and breadth that is substantially similar (for example 3 mm to 10 mm, or 4 mm to 6 mm), and a thickness that is smaller than the length (for example 0.5 mm to 3 mm). In other embodiments the length of the patch may be different (e.g. at least double or triple, or less than half or a quarter) the width of the patch.

The optical fibre 102 is normal to the thickness dimension of the patch, and substantially parallel to the two opposing largest faces of the patch. The optical fibre is preferably embedded in the centre of the patch (i.e. approximately equidistant from the two opposing largest faces). The patch 101 comprises a material with a Poisson's ratio greater than zero, so that extension in length and breadth occurs when the patch 101 is subject to strain in the thickness dimension as a result of a transverse load 110. A reduction in thickness of the patch 101 due to a pressure load on the opposing largest faces of the patch 101 results in an increase in the length and width of the patch 101, as a result of the well-known Poisson effect. For epoxy resins, Poisson's ratio may typically be in the range of 0.35 to 0.42. Since the fibre 102 is embedded in the patch 101, the patch 101 transfers longitudinal strain to the fibre 102 within the patch 101 in response to the transverse pressure load.

The patch 101 may be between 1.2 and 2.5 times the length of the FBG that is embedded therein.

The Young's modulus of the patch material may be varied to provide a different dynamic range and pressure sensitivity of the sensor. The relationship between the Young's modulus of the patch 101 and the coupling of transverse load (or pressure) to extension of the optical fibre 102 may be determined by finite element analysis of the patch and optical fibre. A more flexible patch material will deform more under a specific pressure load, but the reduced stiffness of the patch material may mean that the patch struggles to deform the relatively stiff optical fibre. The patch 101 may be any shape—circular and square patches have been shown to produce good results, but any 3D shape including cylindrical shapes may be used.

The fibre 102 comprises a core 103 and cladding 105. A difference in refractive index between the core 103 and cladding 105 means that light is confined within the core 103 by total internal reflection at the interface between core and cladding.

The patch 101 is configured to provide reduced responsiveness of the embedded FBG to bending (e.g. when the patch is integrated with a textile), primarily by use of a small patch, so that the patch is relatively stiff in bending. The patch 101 therefore prevents bending of the FBG 102.

FIG. 2 illustrates the spectral response of an FBG when unloaded 152 and when subject to a longitudinal (or axial) load 151. The Bragg wavelength has increased in response to the axial load on the fibre, which has increased the grating period by extending the fibre.

FIG. 3 illustrates the relationship between the Bragg wavelength and an applied transverse pressure load on the patch. Data points 171 are shown indicating each measurement of the Bragg wavelength at different pressures. A linear fit 172 is also shown, which is a very good match for the data: the response is very linear.

An optical fibre may be formed with a plurality of Bragg gratings. Patches (as described above) may be formed around at least some of these gratings to form pressure transducers (e.g. a one dimensional array of pressure transducers along the fibre). A grating that is embedded in a patch and which is thereby made responsive to pressure may be termed a transducer FBG, and a patch for rendering an FBG sensitive to pressure may be termed a transducer patch.

Others of the Bragg gratings may be used as temperature and strain reference gratings. An FBG will also respond to temperature—thermal expansion can change the grating period and the refractive index of the materials comprising the FBG. In order to compensate for changes in Bragg wavelength in response to temperature variation, an FBG of the optical fibre can be used as a temperature reference. The temperature reference FBG should preferably be insensitive to strain, so should ideally be protected from extension and bending within a stiff support structure. A further FBG can be used to compensate for tension within the optical fibre, for example resulting from extension of the fibre due to stretching of a textile within which the optical fibre is integrated. As the Young's modulus of the optical fibre 102 is likely to be higher than that of the patch material, any longitudinal force applied to the optical fibre 102 in the vicinity of the patch 101 will result in strain along the transducer FBG within the transducer patch. A strain compensation FBG may be used to compensate for this effect.

FIG. 4 illustrates an example embodiment in which the optical fibre 102 includes a temperature compensation FBG 127, a transducer FBG 107 and a strain compensation FBG 117.

The transducer FBG 107 is integrated within a transducer patch 101, which couples transverse pressure loads into extensional strain in the transducer FBG 107 (as already described).

The temperature compensation FBG 127 is near to (e.g. within 2 cm, 1 cm, or 0.5 mm) the end of the optical fibre 102 and surrounded by a rigid sleeve. The sleeve may be any stiff material such as a metal (e.g. steel). A hypodermic needle or similar may be used as a sleeve. The sleeve is retained over the optical fibre 102 by a patch 111. There is clearance between the sleeve and optical fibre 102, so that the optical fibre remains free to extend and contract due to thermal expansion within the sleeve. The sleeve therefore prevents loading and bending of the end of the fibre 102, while allowing it to respond naturally to temperature changes (as if it were uncoupled from any structure). The patch 111 may have the same properties as the transducer patch 101 around the (or each) transducer FBG 107, so that thermal conductivity and insulation for the temperature compensation FBG 127 is as representative as possible of conditions at the transducer FBG(s) 107.

The strain compensation FBG 117 is not supported by a patch, so is not rendered sensitive to pressure. The strain compensation FBG 117 is responsive to extension of the fibre 102 as a result of tension forces (and may also be subject to changes in grating period arising from bending of the optical fibre 102) and to temperature.

The change in Bragg wavelength for the temperature compensation FBG 127 can be attributed entirely to temperature response. The change in Bragg wavelength for the strain compensation FBG 117 will be due to a combination of temperature response and strain response. The reading from the temperature compensation FBG 127 can be used to remove the temperature response from the strain compensation FBG 117, to leave the strain response only. The strain and temperature response can be used to compensate the response of the transducer FBG 107. One way to do this is to simply subtract the change in wavelength resulting from strain and temperature from the observed change in wavelength at the transducer FBG 107. More complex compensation schemes may also be used, in which a model of the strain and temperature response of the transducer FBG 107 is used to derive a correction from the temperature and strain inferred from the compensation FBGs 117, 127. In some embodiments, the compensation for both temperature and strain can be determined from the corresponding strain compensation FBG only.

The optical fibre 102 may comprise ‘n’ transducer FBGs, and a corresponding plurality of ‘n’ strain compensation FBGs. Each strain compensation FBG may be positioned near to the corresponding transducer FBG (e.g. within less than 1 cm or within less than 2 cm), so that the strain compensation FBG can provide a strain correction that is more closely matched to the strain conditions at the corresponding transducer FBG.

FIG. 5 illustrates an example embodiment in which there are two transducer FBGs 107a, 107b, each embedded within a respective patch 101a, 101b. A single temperature compensation FBG 127 is provided at the end of the optical fibre 102 (as previously described with reference to FIG. 4). Strain compensation FBGs 117a, 117b are provided adjacent to each transducer FBG 107a, 107b. Although an example with only two transducer FBGs is shown, it will be understood that a larger number of transducer FBGs 107 may be used on a single optical fibre (e.g. at least 5, at least 10, at least 20 or at least 50).

Each FBG in an optical fibre may have a slightly different Bragg frequency, so that the response of each FBG in a fibre may be simultaneously determine from the spectral response of the fibre. Provided each FBG has a sufficiently distinct Bragg wavelength it will be straightforward to identify which reflectance peak corresponds with which FBG in the fibre.

A pressure sensor according to an embodiment (e.g. as described above) may be integrated with a textile, for example by knitting, weaving, embroidering or threading the fibre through the textile, or more simply by attaching the fibre to the textile, for example using adhesive, stitching (e.g. tacking stitches with a further yarn), or fixing with heat fusible yarn. A pressure sensor according to an embodiment may be integrated into a textile by embroidering a channel onto the surface of the textile, and then fixing the pressure sensor in the channel, for instance with adhesive and/or heat fusible yarn. At least one transducer FBG and strain compensation FBG may be positioned between each two points of attachment with the fabric.

One configuration of textile and pressure sensing optical fibre is a textile formed into a tube, into which the pressure sensing optical fibre is inserted. If the textile comprises heat fusible yarn, the pressure sensing optical fibre may be fixed in place by fusing the yarn at a number of locations along the fibre to form points of attachment with the optical fibre.

In some embodiments the patch may be formed from a heat fusible yarn. For example, the optical fibre may be woven or knitted into a textile comprising heat fusible yarns (or regions of heat fusible material). The patch structure may be formed around the optical fibre after the fibre has been woven or knitted into the textile by fusing the fibres in the region of a transducer FBG 107. In some embodiments the patch may be formed by impregnating a curable material into a textile in the region of an optical fibre that is woven or knitted (or otherwise integrated) with the textile.

In some embodiments the optical fibre 102 may comprise a high birefringent (‘HiBi’) optical fibre, having two Bragg reflection peaks: one corresponding with horizontally polarized light, and the other corresponding with vertically polarised light. When a transverse load is applied to a HiBi transducer FBG 107 (embedded in a transducer patch 101) the Bragg reflection peaks will separate (or their degree of separation will change)—the response of the peak corresponding with horizontally polarised light will be different to the response of the peak corresponding with vertically polarised light. When a shear stress is applied to the transducer patch 101, the wavelength of both peaks will be affected equally. This is depicted in FIGS. 6 and 7, which respectively illustrate the response of a HiBi transducer FBG 107 in response to a transverse load and a pressure.

FIGS. 6 and 7 show a reflectance peak corresponding with horizontally polarised light 155 and a reflectance peak 156 corresponding with vertically polarised light. As indicated by arrows 159, 160 (in FIG. 6) the effect of a transverse load is to increase the wavelength separation between the reflectance peaks 155, 156. The effect of a shear stress is to increase the wavelength separation between the reflectance peaks 155, 156, as shown by the arrows 157, 158 (in FIG. 7) which illustrate both wavelengths peaks increasing in wavelength in response to a shear load.

FIG. 8 illustrates a characterisation arrangement that has been used to determine the response of transducer FBGs 107 to transverse loading. The patch 101 in which the transducer FBG 107 is embedded is placed between a support 202 and a load 201 (e.g. a weight). The optical fibre 102 is connected to an interrogator 203, which determines the spectral response of the fibre (and FBG in the patch 101). The interrogator outputs data derived from the spectral response to a computer, which is configured to use the data to determine a relationship between the applied load and the response of the transducer FBG in the patch 101.

A number of applications exist for a textile comprising an FBG based pressure sensor. One example application, already discussed above, is in compression bandages. A sensor according to an embodiment may be integrated with a textile bandage to provide a plurality of pressure measurement locations along the length of the bandage. Significantly, pressure sensors according to an embodiment are relatively insensitive to bending, and may include compensation for both temperature and longitudinal strain, which may arise from stretching of the fabric of the bandage. Embodiments in which the transducer FBG is a HiBi FBG can additionally measure shear stress at the patch, which is also likely to have a role in optimal healing.

A sensor according to an embodiment may also be used in other applications. For example, compression garments such as those used in recovery from sports, such as T-shirts, shorts, leggings etc.

A sensor according to an embodiment may be used to determine a dynamic stiffness index of a compression bandage or compression garment.

In addition to measuring a relatively steady state compression, as in the case of a compression bandage or compression garment, a sensor according to an embodiment may be used to measure more transient pressure loading, such as may result from impact. For example, sensors according to an embodiment may be integrated with an upper or lower of a shoe, or within a sock, to measure pressure and shear stress distribution around a foot when moving (e.g. walking, running, participating in sporting activity). Sensors under the foot can be useful in gait analysis, and information from such sensors can subsequently be used in a diagnostic or therapeutic context. Data obtained from sensors around a foot can also be used to inform the design of better footwear and/or orthoses, in a way that is specific to a particular user and/or their particular mode of use of the footwear. For example, positions of high stress or shear can be identified, and the design of the orthosis or footwear modified to reduce or eliminate such. Positions of high pressure or shear may be associated with tissue breakdown.

With a suitable portable interrogator and data storage arrangement, a wearable sensor may be used during sporting activity. For example, a football player may wear a sock comprising an array of pressure sensors according to an invention in order to identify at least one of ways to improve their performance at the sport, and ways to improve the performance of their footwear/orthotics. It will be appreciated that the invention can be applied to footwear design in relation to any sport, and is not restricted to sports in which the feet are used to strike objects (such as rugby, football, etc). For example, running shoes that are optimised based on pressure/shear data obtained during use may provide more comfort and/or appropriate support, and therefore enhanced performance.

An alternative application for a pressure sensor according to an embodiment is in chairs or seat for improving posture while sitting, for instance at a workstation or driving. Embodiments of the invention may be integrated with a chair or seat at relatively low cost, and provide an array of pressure measurements. Data derived from the sensors may be used to suggest improvements to seating posture, for example by way of a message on the dashboard of a car indicating that a shift in posture is likely to improve comfort.

A pressure sensor according to an embodiment may be used in a mattress, to indicate how tissue should be unloaded, or how to adjust the mattress, for example to prevent bedsores in bed-bound patients.

Although a number of example embodiments have been described, a number of modifications and variations are possible, and the scope of the invention should be determined with reference to the appended claims.

What is claimed is:

1-27. (canceled)

28. A textile comprising:
a pressure sensor, the pressure sensor comprising an optical fibre with at least one transducer FBG (fibre Bragg grating), and at least one transducer patch having a first face and a second face opposite the first face;
wherein the transducer FBG is embedded in the transducer patch;
wherein the transducer patch is less than 10 mm in length along the optical fibre; and
wherein the transducer patch is configured to cause longitudinal strain in the transducer FBG in response to the transducer patch being subject to a pressure load on the first face and the second face, the longitudinal strain arising as a result of extension of the transducer patch due to a Poisson effect.

29. The textile of claim 28, wherein the transducer patch:
is 4 mm to 6 mm in length along the optical fibre;
has a width of less than 10 mm; and/or
has a thickness of 0.5 mm to 3 mm.

30. The textile of claim 28, wherein the optical fibre further comprises a strain compensation FBG that is not embedded in a patch, for providing compensation for longitudinal force applied to the optical fibre.

31. The textile of claim 28, wherein the optical fibre comprises a temperature compensation FBG and pressure insensitive patch, wherein:
the temperature compensation FBG is a last FBG before an end of the optical fibre;
the pressure insensitive patch comprises a substantially rigid support member with a cavity; and
the temperature compensation FBG is received in a clearance fit within the cavity so that the temperature compensation FBG is free to expand and to contract independently of the rigid support member.

32. The textile of claim 28, wherein the at least one transducer patch of the pressure sensor comprises a plurality of transducer patches, and the at least one transducer FBG of the optical fibre comprises a plurality of transducer FBGs; and
wherein each transducer FBG of the plurality of transducer FBGs is embedded in a respective transducer patch of the plurality of transducer patches, so as to provide a pressure sensor with a plurality of pressure transducers along the optical fibre.

33. The textile of claim 32, wherein the pressure sensor further comprises a plurality of strain compensation FBGs, wherein each strain compensation FBG of the plurality of strain compensation FBGs is not embedded in a patch and is configured to provide strain compensation for longitudinal force applied to the optical fibre, and each transducer FBG of the plurality of transducer FBGs has a corresponding strain compensation FBG of the plurality of strain compensation FBGs adjacent thereto.

34. The textile of claim 28, wherein the optical fibre is a high birefringent optical fibre, so that the transducer FBG has a spectral response that enables pressure to be distinguished from shear stress.

35. The textile of claim 28, wherein the textile comprises a heat fusible yarn, and the optical fibre is attached to a fused region of the heat fusible yarn, or wherein the optical fibre is integrated with the textile by knitting, weaving, embroidering or threading the optical fibre into the textile.

36. The textile of claim 28, wherein the textile comprises at least a portion of a compression bandage, a compression garment, a sock, footwear or orthotic.

37. A system for determining pressure on a textile in at least one location, comprising:
a textile comprising a pressure sensor, the pressure sensor comprising an optical fibre with at least one transducer FBG (fibre Bragg grating), and at least one transducer patch having a first face and a second face opposite the first face;
wherein the transducer FBG is embedded in the transducer patch;
wherein the transducer patch is less than 10 mm in length along the optical fibre; and
wherein the transducer patch is configured to cause longitudinal strain in the transducer FBG in response to the transducer patch being subject to a pressure load on the first face and the second face, the longitudinal strain arising as a result of extension of the transducer patch due to a Poisson effect;
wherein the optical fibre further comprises a strain compensation FBG that is not embedded in a patch, for providing compensation for longitudinal force applied to the optical fibre, and
an optical interrogator for determining a Bragg wavelength of the transducer FBG and strain compensation FBG; and
a processor configured to determine a pressure load from the Bragg wavelength of the transducer FBG, including compensating for longitudinal force applied to the optical fibre.

38. The system of claim 37, wherein compensating for the longitudinal force applied to the optical fibre comprises subtracting a wavelength shift of the strain compensation FBG from a wavelength shift of the transducer FBG before determining the pressure load, the textile further comprising a temperature compensation FBG and pressure insensitive patch, wherein:
the temperature compensation FBG is a last FBG before an end of the optical fibre, the pressure insensitive patch comprises a substantially rigid support member with a cavity, the temperature compensation FBG is received in a clearance fit within the cavity so that the temperature compensation FBG is free to expand and to contract independently of the rigid support member, and
the processor is configured to compensate for temperature using the Bragg wavelength of the temperature compensation FBG when determining the pressure load.

39. A method of monitoring pressure applied to a portion of a user's body via a textile, comprising:
applying the textile to the user's body, the textile comprising a pressure sensor, the pressure sensor comprising an optical fibre with at least one transducer FBG (fibre Bragg grating), and at least one transducer patch having a first face and a second face opposite the first face;
wherein the transducer FBG is embedded in the transducer patch;
wherein the transducer patch is less than 10 mm in length along the optical fibre; and
wherein the transducer patch is configured to cause longitudinal strain in the transducer FBG in response to the transducer patch being subject to a pressure load on the first face and the second face, the longitudinal strain arising as a result of extension of the transducer patch due to a Poisson effect; and
using an optical interrogator to determine a Bragg wavelength of the at least one transducer FBG while the textile is in contact with the user's body,
using a processor to determine at least one pressure load from the Bragg wavelength from the at least one transducer FBG.

40. The method of claim 39, wherein applying the textile to the user's body comprises at least one of a user: wearing the textile, lying on the textile, sitting on the textile, resting part of their body on the textile, and/or performing a work or leisure activity while at least part of their body is in contact with the textile.

41. The method of claim 39, further comprising using the optical interrogator to determine a Bragg wavelength of at least one strain compensation FBG and/or at least one temperature compensation FBG; and
using the processor to determine a pressure load from the Bragg wavelength of the transducer FBG, includes compensating for longitudinal force applied to the optical fibre.

42. The method of claim 41, wherein compensating for a longitudinal force applied to the optical fibre comprises subtracting the wavelength shift of the strain compensation FBG from the wavelength shift of the transducer FBG before determining the pressure load.

43. The method of claim 42, the textile further comprising a temperature compensation FBG and pressure insensitive patch, wherein:
the temperature compensation FBG is the last FBG before the end of the optical fibre, the pressure insensitive patch comprises a substantially rigid support member with a cavity, the temperature compensation FBG is received in a clearance fit within the cavity so that the temperature compensation FBG is free to expand and contract independently of the rigid support member, and
the processor compensates for temperature using the Bragg wavelength of the temperature compensation FBG when determining the pressure load.

44. The method of claim 39, wherein the textile comprises as at least a portion of a sock, footwear, orthotic or prosthesis; and
the user performing an activity while wearing the sock, the footwear, the orthotic or the prosthesis;
recording the at least one pressure load, the at least one pressure load indicating at least pressure in at least one location of the user;
modifying a design of the sock, the footwear, the orthotic or the prosthesis in response to the recording of the at least one pressure load.

45. The method of claim 44, wherein modifying the design comprises identifying at least one region of high pressure, and modifying the design of the sock, the footwear, the orthotic or the prosthesis to reduce pressure in the high pressure region.

46. The method of claim 44, wherein the activity is a sporting activity.

47. The method of claim 39, wherein the textile comprises at least a portion of a compression bandage; and
using the optical interrogator to determine the interface pressure of the compression bandage in at least one location from the Bragg wavelength of at least one transducer FBG.
